(19) United States
(12) Patent Application Publication
(10) Pub. No.: US 2021/0154110 A1
(43) Pub. Date: May 27, 2021

(54) COSMETIC EXCIPIENT INCLUDING A C8-C10 ALKANE AND A C>= 11 ALKANES

(71) Applicant: BIOSYNTHIS, Saint Cyr Sous Dourdan (FR)

(72) Inventor: Thierry BERNOUD, Saint Cyr Sous Dourdan (FR)

(73) Assignee: BIOSYNTHIS, Saint Cyr Sous Dourdan (FR)

(21) Appl. No.: 17/047,136

(22) PCT Filed: Apr. 10, 2019
(86) PCT No.: PCT/EP2019/059172

§ 371 Date: Oct. 13, 2020

(30) Foreign Application Priority Data
Apr. 10, 2018 (FR) 1853142

(51) Int. Cl. A61K 8/31; A61Q 1/14; A61Q 19/00; A61Q 1/04; A61K 8/58; A61Q 5/12

(52) U.S. Cl. CPC A61K 8/31; A61Q 1/14; A61Q 19/001; A61Q 1/04; A61K 2800/92; A61Q 5/12; A61K 2800/54; A61K 2800/5922; A61K 8/585

(57) ABSTRACT

A cosmetic excipient including an alkane mixture, the alkane mixture including at least one C8-C10 alkane, and at least one C≥11 alkane, wherein the at least one C8-C10 alkane is present in a percentage by mass less than or equal to approximately 40% (≤40%), relative to the total mass of the alkane mixture.

The invention relates to the technical field of cosmetic care for the skin and integuments.

More specifically, the invention relates to a cosmetic excipient, as well as a cosmetic formulation.

A number of definitions must be given.

The term “alkane” is used to denote a saturated hydrocarbon consisting only of carbon and hydrogen atoms linked together by single covalent bonds, the general formula of which is CₙH₂ₙ₊₂.

The term “linear alkane” is used to refer to an alkane in which each carbon atom is bound to a maximum of two carbon atoms.

The term “branched alkane” is used to refer to an alkane in which certain carbon atoms are bound to three or even four carbon atoms.

The term “Cₓ alkane” is used to denote an alkane consisting of x carbon atoms; said alkane may be linear or branched. For example, n-decane is a C10 alkane.

An alkane consisting of a carbon number of x or less than x is called “C≤x alkane”. For example, a C≤2 alkane may be C1 or C2.

The term “C≥x alkane” is used to denote an alkane consisting of a carbon number of x or more than x. For example, a C≥2 alkane may be any alkane, excluding methane (C1).

The term “Cₓ-C_{y} alkane” is used to denote an alkane consisting of x to y carbon atoms; said alkane may be linear or branched. For example, the C1-C2 alkanes are methane and ethane.

The term “methylalkane” is used to refer to a branched alkane comprising at least one branch being a methyl group. For example, 4-methylnonane, or even 2,3-dimethyloctane, are methylalkanes, and more precisely C10 methylalkanes. For example, methylundecanes are C12 methylalkanes. For example, dimethyloctanes are C12 methylalkanes. Methylheptadecanes are C18 alkanes. These methylalkanes are preferably obtained by dehydration of a Guerbet alcohol. When asymmetric carbons are present, the term branched alkanes is understood to be racemic mixtures and separate isomers.

The term “deodorized alkane” refers to an alkane which has undergone a deodorization operation, said deodorization making it possible to remove the impurities responsible for an unpleasant odor for the user. These impurities can in particular be residues of the Guerbet alcohol, which are the most commonly used starting materials. These very low rates are obtained thanks to a very high dehydration yield. A deodorized alkane according to the invention is an alkane which comprises less than 0.01% of the corresponding Guerbet alcohol residue.

A deodorized alkane is moreover free from sulfur compounds such as thiols, for example 1-nonanethiol; The term “free” is understood to mean a content of less than 0.01%.

The flash point measured according to ASTM D93 standard is called “flash point”. The flash point unit as measured according to the ASTM D93 standard is degrees Celsius (° C.). It may also be an average of values, each of the values being obtained by means of a measurement according to ASTM D93.

The term “percentage of evaporation at t=X” refers to the percentage of compound (or mixture of compounds) evaporated at a given time (X), the evaporation conditions being the following: LABOMODERNE THERMOBALANCE KERN DBS 60-3 desiccator, 20° C., under controlled atmosphere. For example, if the evaporation percentage is at least 60% after 3 hours, it means that at least 60% of the compound (or mixture of compounds) has evaporated after 3 hours.

The term “percentage by mass” refers to the ratio of the mass of a first compound relative to the total mass of a mixture of compounds (comprising the first compound) or composition, reduced to a percentage. For example, if 10 grams of a compound are present in a mixture z having a total mass of 100 grams, then the percentage by mass of y in z is 10%.

The term “percentage by weight” refers to the ratio of the weight of a constituent relative to the total weight of the composition, reduced to 100.

In the context of this application, the term “approximately” preceding a numerical value means that the value may be modified by +/−10%. In the particular case of a numerical value being an interval limit, the term “approximately” means that the lower limit may be reduced by 10% or the upper limit may be increased by 10%. It is also possible to delete the term “approximately” preceding a numerical value.

Since the appearance of silicones in cosmetics in the 1950s, their use has expanded considerably in all fields of cosmetics, to include skin care, makeup and hair products. This wide use is explained by the specific physicochemical characteristics of silicones. There are 5 main categories of silicones: volatile silicone oils, non-volatile silicone oils, modified silicone oils, silicone waxes, as well as silicone gums.

Volatile silicone oils are very often composed of cyclic silicones with short chains of the general formula [Si—(CH₃)₂—O]ₙ with n being from 3 to 7 (limits included). They are generally called cyclomethicones, regardless of the number of units. The most common cyclomethicones are cyclotetrasiloxane (D4) (C₈H₂₄O₄Si₄, CAS 556-67-2), cyclopentasiloxane (D5) (C₁₀H₃₀O₅Si₅, CAS 541-02-6) and cyclohexasiloxane (D6) (C₁₂H₃₆O₆Si₆, CAS 540-97-6).

Cyclomethicones have, until very recently, been regarded as harmless skin emollients and solvents (see for example, International Journal of Toxicology, Volume 10, No. 1, pp. 9-19 1991); it is only relatively recently that their potential deleterious effects on the environment, and even on human health, have been demonstrated.

Research has therefore been carried out in order to identify compounds likely to offer a satisfactory alternative, these compounds having to have similar volatility characteristics and a similar behavior in a formulation, in particular in terms of viscosity and sensory properties (ability to spread on the skin and for the film obtained to feel soft, “dry” and non-greasy).

The sensory properties are tested by panels, called sensory panels, see for example, Concepton of cosmetic products: formulation, Anne-Marie PENSE-LHERITIER, page 95, LAVOISIER.

For example, on behalf of COGNIS, application US20050079986 has proposed an oily composition containing a mixture of a linear or branched dialkyl carbonate with a preferably branched and saturated alkane, containing from 8 to 40 carbon atoms.

On behalf of INOLEX, application US20040241200 has also suggested using a combination of isoparaffin such as isododecane with a neopentylglycol polyester, such as neopentylglycol diheptanoate. This mixture is marketed in particular by the company INOLEX under the trade name LexFeel® D4 and D5. PRESPERSE LLC also offers, under the trade name SiClone® SR-5, a mixture of C₁₃-C₁₆, C₁₂-C₁₄ isoparaffins with C₁₃-C₁₅ alkanes.

In addition, on behalf of BERNEL, U.S. Pat. No. 6,126,951 has proposed the use of a caprylyl isostearate resulting from the esterification of 2-octanol by a mixture of C₁₈ fatty acid isomers, without, however, achieving the volatility sought.

Finally, on behalf of the Applicant, application WO2010115973 has proposed using a alkane mixture.

However, the need remains to have a cosmetic excipient having physicochemical and sensory characteristics even closer to those of cyclomethicones and capable of replacing them, in particular as an excipient to be included in cosmetic formulations.

Surprisingly, it has been demonstrated by the Applicant that an ideal cosmetic excipient with regard to the objectives identified above could be obtained.

Surprisingly, it has been demonstrated that within a alkane mixture, the C8-C10 alkanes (and more particularly C10) have a particular and unpredictable influence on the volatility properties of the alkane mixture (being specifically noted by measuring evaporation and flash point).

For example, an alkane mixture comprising 30% n-decane (C10, flash point 46° C.), 40% n-dodecane (C12, flash point 71° C.) and 30% n-tetradecane (C14, flash point 115° C.) has a flash point of 66° C., which was not predictable.

In a particularly advantageous embodiment, the cosmetic excipient according to the invention comprises a alkane mixture, said mixture having a flash point of less than 69° C., preferably between 60 and 69° C., preferably between 60 and 67° C., preferably between 62 and 65° C.

It has also been demonstrated that the use of a cosmetic excipient consisting only of branched alkanes makes it possible to obtain more stable emulsions than the emulsions obtained with linear alkanes.

It was also demonstrated that the inclusion of a cosmetic excipient according to the invention in cosmetic formulations, had the effect of providing said cosmetic formulation with remarkable cosmetic qualities in addition to excellent stability in the emulsions such as better dispersibility, i.e., the possibility of obtaining regular dispersions, of powders, for example pigments, and better spreadability of the compositions on the skin, these properties being particularly important for make-up products, such as foundations and sunscreens.

The invention relates to a cosmetic excipient comprising a alkane mixture, said alkane mixture comprising at least one C8-C10 alkane, and at least one C alkane ≥11, characterized in that said at least one C8-C10 alkane is present in a percentage by mass of less than or equal to approximately 50% (≤50%), relative to the total mass of said alkane mixture.

In one embodiment, said alkane mixture is a alkane mixture whose number of atoms is even.

In one embodiment, said alkane mixture is exempt of alkanes whose number of carbon atoms is odd.

This parity is due to the natural origin of the alcohols which are used as starting products whether for the synthesis of Guerbet alcohols or which are dehydrated to obtain said alkanes, in fact natural alcohols contain an even number of carbon atoms.

The invention also relates to a cosmetic excipient comprising a alkane mixture as defined above, characterized in that said at least one C8-C10 alkane is chosen from the group consisting of C8 and C10 alkanes, and mixtures thereof.

In one embodiment, the cosmetic excipient according to the invention is characterized in that said at least one C8-C10 alkane is chosen from the group consisting of linear or branched C8 and C10 alkanes, and mixtures thereof.

In one embodiment, the cosmetic excipient according to the invention is characterized in that said at least one C8-C10 alkane is selected from the group consisting of linear C8 and C10 alkanes, and mixtures thereof.

In one embodiment, the cosmetic excipient according to the invention is characterized in that said at least one C8-C10 alkane is selected from the group consisting of branched C8 and C10 alkanes, and mixtures thereof.

In one embodiment, the cosmetic excipient according to the invention is characterized in that said at least one C8-C10 alkane is at least one C10 alkane.

In one embodiment, the cosmetic excipient according to the invention is characterized in that said at least one C8-C10 alkane is at least a linear or branched C10 alkane.

In one embodiment, the cosmetic excipient according to the invention is characterized in that said at least one C8-C10 alkane is at least one linear C10 alkane.

In one embodiment, the cosmetic excipient according to the invention is characterized in that said at least one C8-C10 alkane is at least one branched C10 alkane.

In one embodiment, the cosmetic excipient according to the invention is characterized in that said at least one C8-C10 alkane is at least one branched C10 alkane selected from the group consisting of methylnonanes, such as, for example, 2-methylnonane (CAS 871-83-0), ethyloctanes, such as, for example, 3-ethyloctane (CAS 5881-17-4), dimethyloctanes, such as, for example, 2.2-dimethyloctane (CAS 15869-87-1), propylheptanes, such as, for example, 4-propylheptane (CAS 3178-29-8), ethylmethylheptanes, such as, for example, 3-ethyl-2-methylheptane (CAS-14676-29-0), trimethylheptanes, such as, for example, 2.2-trimethylheptane (CAS 52896-92-1), diethylhexanes, such as, for example, 3.3-diethylhexane (CAS 17302-02-2), tetramethylhexanes, such as, for example, 2,2,3,3-tetramethylhexane (CAS 13475-81-5), ethyltrimethylpentanes, such as, for example, 3-ethyl-2,2,3-trimethylpentane (CAS 52897-17-3), and mixtures thereof.

In one embodiment, the cosmetic excipient according to the invention is characterized in that said at least one C8-C10 alkane is at least one C10 methyl alkane.

In one embodiment, the cosmetic excipient according to the invention is characterized in that said at least one C8-C10 alkane is chosen from the group consisting of 4-methylnonane (C10H22, CAS 17301-94-9), 2-methylnonane (C10H22, CAS 871-83-0), n-decane (C10H22, CAS 124-18-5), and mixtures thereof.

In one embodiment, the cosmetic excipient according to the invention is characterized in that said at least one C8-C10 alkane is at least 4-methylnonane.

In one embodiment, the cosmetic excipient according to the invention is characterized in that said at least one C8-C10 alkane is at least 2-methylnonane.

In one embodiment, the cosmetic excipient according to the invention is characterized in that said at least one C8-C10 alkane is at least n-decane. Particularly preferred is n-decane.

The invention also relates to a cosmetic excipient comprising a alkane mixture as defined above, characterized in that said at least one C8-C10 alkane is present in a percentage by mass from approximately 1 to approximately 50% (1≤percentage by mass≤50%), relative to the total mass of said alkane mixture.

The invention also relates to a cosmetic excipient comprising a alkane mixture as defined above, characterized in that said at least one C8-C10 alkane is present in a percentage by mass from approximately 1 to approximately 40% (1≤percentage by mass≤40%), relative to the total mass of said alkane mixture.

In one embodiment, the cosmetic excipient according to the invention is characterized in that said at least one C8-C10 alkane is present in a percentage by mass from approximately 1 to approximately 30% (1%≤percentage by mass≤30%), relative to the total mass of said alkane mixture.

In one embodiment, the cosmetic excipient according to the invention is characterized in that said at least one C8-C10 alkane is present in a percentage by mass from approximately 5 to approximately 30% (5%≤percentage by mass≤30%), relative to the total mass of said alkane mixture.

In one embodiment, the cosmetic excipient according to the invention is characterized in that said at least one C8-C10 alkane is present in a percentage by mass from approximately 10 to approximately 30% (10%≤percentage by mass≤30%), relative to the total mass of said alkane mixture.

In one embodiment, the cosmetic excipient according to the invention is characterized in that said at least one C8-C10 alkane is present in a percentage by mass from approximately 15 to approximately 30% (15%≤percentage by mass≤30%), relative to the total mass of said alkane mixture.

The invention also relates to a cosmetic excipient comprising a alkane mixture as defined above, characterized in that said at least one C≥11 alkane is selected from the group consisting of C12, C14, C16, C18, C20, C22, C24 alkanes, and mixtures thereof.

In one embodiment, the cosmetic excipient according to the invention is characterized in that said at least one C≥11 alkane is selected from the group consisting of linear and branched C12, C14, C16, C18, C20, C22, C24 alkanes, and mixtures thereof.

In one embodiment, the cosmetic excipient according to the invention is characterized in that said at least one C alkane≥11, is selected from the group consisting of C12, C14, C16, C18, C20, C22, C24 linear alkanes, and mixtures thereof.

In one embodiment, the cosmetic excipient according to the invention is characterized in that said at least one C≥11 alkane is selected from the group consisting of branched C12, C14, C16, C18, C20, C22, C24 alkanes, and mixtures thereof.

In one embodiment, the cosmetic excipient according to the invention is characterized in that said at least one C≥11 alkane is selected from the group consisting of n C12, C14, C16, C18, C20, C22, C24 alkanes, and mixtures thereof.

In one embodiment, the cosmetic excipient according to the invention is characterized in that said at least one C≥11 alkane is selected from the group consisting of linear or branched C12, C14, C16, C18, C20, C22, C24 alkanes, and mixtures thereof.

In one embodiment, the cosmetic excipient according to the invention is characterized in that said at least one C≥11 alkane is selected from the group consisting of linear C12, C14, C16, C18, C20, C22, C24 alkanes, and mixtures thereof.

In one embodiment, the cosmetic excipient according to the invention is characterized in that said at least one C≥11 alkane is selected from the group consisting of branched C12, C14, C16, C18, C20, C22, C24 alkanes, and mixtures thereof.

In one embodiment, the cosmetic excipient according to the invention is characterized in that said at least one C≥11 alkane is selected from the group consisting of C12, C14, C16 alkanes, and mixtures thereof.

In one embodiment, the cosmetic excipient according to the invention is characterized in that said at least one C≥11 alkane is selected from the group consisting of linear or branched C12, C14, C16 alkanes, and mixtures thereof.

In one embodiment, the cosmetic excipient according to the invention is characterized in that said at least one C≥11 alkane is selected from the group consisting of linear or branched C12 alkanes (selected from the group consisting of 5-methylundecane (C12H26, CAS 1632-70-8) 2-methylnonane (C12H26, CAS 31807-55-3), n-dodecane (C12H26, CAS 112-40-3), and mixtures thereof), C14 alkanes (chosen from the group consisting of 6-methyltridecane (C14H30, CAS 13287-21-3), 2-methyltridecane (C14H30, CAS 1560-96-9), n-tetradecane (C14H30, CAS 629-59-4), and mixtures thereof), C16 alkanes (being isohexadecane (C16H34, CAS 93685-80-4)), and mixtures thereof.

In one embodiment, the cosmetic excipient according to the invention is characterized in that said at least one C≥11 alkane is selected from the group consisting of linear C12, C14, C16 alkanes, and mixtures thereof.

In one embodiment, the cosmetic excipient according to the invention is characterized in that said at least one C≥11 alkane is selected from the group consisting of branched C12, C14, C16 alkanes, and mixtures thereof.

In one embodiment, the cosmetic excipient according to the invention is characterized in that said at least one C≥11 alkane is at least a C12 alkane.

In one embodiment, the cosmetic excipient according to the invention is characterized in that said at least one C≥11 alkane is at least a linear or branched C12 alkane.

In one embodiment, the cosmetic excipient according to the invention is characterized in that said at least one C≥11 alkane is at least a linear C12 alkane.

In one embodiment, the cosmetic excipient according to the invention is characterized in that said at least one C≥11 alkane is at least a branched C12 alkane.

In one embodiment, the cosmetic excipient according to the invention is characterized in that said at least one C≥11 alkane is at least a C12 methyl alkane.

In one embodiment, the cosmetic excipient according to the invention is characterized in that said at least one C≥11 alkane is selected from the group consisting of 5-methylundecane (C12H26, CAS 1632-70-8), 2-methylundecane (C12H26, CAS 31807-55-3), n-dodecane (C12H26, CAS 112-40-3), and mixtures thereof.

In one embodiment, the cosmetic excipient according to the invention is characterized in that said at least one C≥11 alkane is at least 5-methylundecane.

In one embodiment, the cosmetic excipient according to the invention is characterized in that said at least one C≥11 alkane is at least 2-methylundecane.

In one embodiment, the cosmetic excipient according to the invention is characterized in that said at least one C≥11 alkane is at least n-dodecane.

In one embodiment, the cosmetic excipient according to the invention is characterized in that said at least one C≥11 alkane is at least a C14 alkane.

In one embodiment, the cosmetic excipient according to the invention is characterized in that said at least one C≥11 alkane is at least a linear or branched C14 alkane.

In one embodiment, the cosmetic excipient according to the invention is characterized in that said at least one C≥11 alkane is at least a linear C14 alkane.

In one embodiment, the cosmetic excipient according to the invention is characterized in that said at least one C≥11 alkane is at least a branched C14 alkane.

In one embodiment, the cosmetic excipient according to the invention is characterized in that said at least one C≥11 alkane is at least a C14 methyl alkane.

In one embodiment, the cosmetic excipient according to the invention is characterized in that said at least one C≥11 alkane is chosen from the group consisting of 6-methyltridecane (C14H30, CAS 13287-21-3), 2-methyltridecane (C14H30, CAS 1560-96-9), n-tetradecane (C14H30, CAS 629-59-4), and mixtures thereof.

In one embodiment, the cosmetic excipient according to the invention is characterized in that said at least one C≥11 alkane is at least 6-m ethyltridecane.

In one embodiment, the cosmetic excipient according to the invention is characterized in that said at least one C≥11 alkane is at least 2-methyltridecane.

In one embodiment, the cosmetic excipient according to the invention is characterized in that said at least one C≥11 alkane is at least n-tetradecane.

In one embodiment, the cosmetic excipient according to the invention is characterized in that said at least one C≥11 alkane is at least a C16 alkane.

In one embodiment, the cosmetic excipient according to the invention is characterized in that said at least one C≥11 alkane is at least a linear or branched C16 alkane.

In one embodiment, the cosmetic excipient according to the invention is characterized in that said at least one C≥11 alkane is at least a linear C16 alkane.

In one embodiment, the cosmetic excipient according to the invention is characterized in that said at least one C≥11 alkane is at least a branched C16 alkane.

In one embodiment, the cosmetic excipient according to the invention is characterized in that said at least one C≥11 alkane is at least a C16 methyl alkane.

In one embodiment, the cosmetic excipient according to the invention is characterized in that said at least one C≥11 alkane is isohexadecane (C16H34, CAS 93685-80-4).

In one embodiment, the cosmetic excipient according to the invention is characterized in that said at least one C12 alkane is branched.

In one embodiment, the cosmetic excipient according to the invention is characterized in that said at least one branched C12 alkane is chosen from the group consisting of 5-methylundecane (CAS 1632-70-8), 2-methylundecane (CAS 31807-55-3), 2,3-dimethyldecane (CAS 17312-44-6), 2,5-dimethyldecane (CAS 17312-50-4), 3-methylundecane (CAS 1002-43-3) and mixtures thereof.

In one embodiment, the cosmetic excipient according to the invention is characterized in that said at least one C14 alkane is branched.

In one embodiment, the cosmetic excipient according to the invention is characterized in that said at least one branched C14 alkane is chosen from the group consisting of 2-methyltridecane (CAS 1560-96-9), 6-methyltridecane (CAS 13287-21-3) and mixtures thereof.

In one embodiment, the cosmetic excipient according to the invention is characterized in that said at least one C16 alkane is branched.

In one embodiment, the cosmetic excipient according to the invention is characterized in that said at least one branched C16 alkane is selected from the group consisting of isohexadecane (CAS 93685-80-4), 2-methylpentadecane (CAS 1560-93-6) and mixtures thereof.

In one embodiment, the cosmetic excipient according to the invention is characterized in that said at least one C≥11 alkane is at least a C20 alkane.

In one embodiment, the cosmetic excipient according to the invention is characterized in that said at least one C≥11 alkane is a branched C20 alkane. In one embodiment, the cosmetic excipient according to the invention is characterized in that said at least one branched C20 is isoeicosane (CAS 52845-07-5).

The invention also relates to a cosmetic excipient comprising a alkane mixture as defined above, characterized in that said at least one C≥11 alkane is present in a percentage by mass of from approximately 50 to approximately 99% (50%≤percentage by mass≤99%), relative to the total mass of said alkane mixture.

The invention also relates to a cosmetic excipient comprising a alkane mixture as defined above, characterized in that said at least one C≥11 alkane is present in a percentage by mass of from approximately 60 to approximately 99% (50%≤percentage by mass≤99%), relative to the total mass of said alkane mixture. In one embodiment, the cosmetic excipient according to the invention is characterized in that said at least one C≥11 alkane is present in a percentage by mass of from approximately 70 to approximately 99% (70%≤percentage by mass≤99%), relative to the total mass of said alkane mixture.

In one embodiment, the cosmetic excipient according to the invention is characterized in that said at least one C≥11 alkane is present in a percentage by mass of from approximately 80 to approximately 99% (70%≤percentage by mass≤99%), relative to the total mass of said alkane mixture.

In one embodiment, the cosmetic excipient according to the invention is characterized in that said at least one C≥11 alkane is present in a percentage by mass of from approximately 70 to approximately 95% (70%≤percentage by mass≤95%), relative to the total mass of said alkane mixture.

In one embodiment, the cosmetic excipient according to the invention is characterized in that said at least one C≥11 alkane is present in a percentage by mass of from approximately 70 to approximately 90% (70%≤percentage by mass≤90%), relative to the total mass of said alkane mixture.

In one embodiment, the cosmetic excipient according to the invention is characterized in that said at least one C≥11 alkane is present in a percentage by mass of from approximately 70 to approximately 85% (70%≤percentage by mass≤85%), relative to the total mass of said alkane mixture.

The invention also relates to a cosmetic excipient comprising a alkane mixture as defined above, characterized in that said at least one C8-C10 alkane is a C10, and the at least one C≥11 alkane is a C12 alkane.

In one embodiment, the cosmetic excipient according to the invention is characterized in that said at least one C8-C10 alkane is a C10 alkane present in said excipient at a percentage by mass from approximately 1 to approximately 50% (1%≤percentage by mass≤50%), relative to the total mass of said alkane mixture and the at least one C≥11 alkane is a C12 alkane present in said excipient at a percentage by mass from approximately 50 to approximately 99% (50%≤percentage by mass≤99%), relative to the total mass of said alkane mixture.

In one embodiment, the cosmetic excipient according to the invention is characterized in that said at least one C8-C10 alkane is a C10 alkane present in said excipient at a percentage by mass from approximately 1 to approximately 30% (1%≤percentage by mass≤30%), relative to the total mass of said alkane mixture and the at least one C≥11 alkane is a branched C12 alkane present in said excipient at a percentage by mass from approximately 70 to approximately 99% (70%≤percentage by mass≤99%), relative to the total mass of said alkane mixture.

In one embodiment, the cosmetic excipient according to the invention is characterized in that said at least one C8-C10 alkane is a C10 alkane present in said excipient at a percentage by mass from approximately 5 to approximately 30% (5%≤percentage by mass≤30%), relative to the total mass of said alkane mixture and the at least one C≥11 alkane is a branched C12 alkane present in said excipient at a percentage by mass from approximately 70 to approximately 95% (70%≤percentage by mass≤95%), relative to the total mass of said alkane mixture.

In one embodiment, the cosmetic excipient according to the invention is characterized in that said at least one C8-C10 alkane is a C10 alkane present in said excipient at a percentage by mass from approximately 10 to approximately 30% (10%≤percentage by mass≤30%), relative to the total mass of said alkane mixture and the at least one C≥11 alkane is a branched C12 alkane present in said excipient at a percentage by mass from approximately 70 to approximately 90% (70%≤percentage by mass≤90%), relative to the total mass of said alkane mixture.

In one embodiment, the cosmetic excipient according to the invention is characterized in that said at least one C8-C10 alkane is a C10 alkane present in said excipient at a percentage by mass from approximately 15 to approximately 30% (15%≤percentage by mass≤30%), relative to the total mass of said alkane mixture and the at least one C≥11 alkane is a branched C12 alkane present in said excipient at a percentage by mass from approximately 70 to approximately 85% (70%≤percentage by mass≤85%), relative to the total mass of said alkane mixture.

In one embodiment, the cosmetic excipient according to the invention is characterized in that said at least one C8-C10 alkane is a C10 alkane, and the at least one C≥11 alkane is a linear C12 alkane.

In one embodiment, the cosmetic excipient according to the invention is characterized in that said at least one C8-C10 alkane is a C10 alkane, and the at least one C≥11 alkane is a branched C12 alkane. The branched C12 alkane is preferred over the linear C12 alkane.

The invention also relates to a cosmetic excipient comprising an alkane mixture as defined above, characterized in that said at least one C8-C10 alkane is a C10, and the at least one C≥11 alkane is a C14 alkane.

In one embodiment, the cosmetic excipient according to the invention is characterized in that said at least one C8-C10 alkane is a C10 alkane present in said excipient at a percentage by mass from approximately 1 to approximately 50% (10%≤percentage by mass≤50%), relative to the total mass of said alkane mixture and the at least one C≥11 alkane is a C14 alkane present in said excipient at a percentage by mass from approximately 50 to approximately 99% (50%≤percentage by mass≤99%), relative to the total mass of said alkane mixture.

In one embodiment, the cosmetic excipient according to the invention is characterized in that said at least one C8-C10 alkane is a C10 alkane present in said excipient at a percentage by mass from approximately 1 to approximately 30% (1%≤percentage by mass≤30%), relative to the total mass of said alkane mixture and the at least one C≥11 alkane is a branched C14 alkane present in said excipient at a percentage by mass of between approximately 70 and approximately 99% (70%≤percentage by mass £ 99%), relative to the total mass of said alkane mixture.

In one embodiment, the cosmetic excipient according to the invention is characterized in that said at least one C8-C10 alkane is a C10 alkane present in said excipient at a percentage by mass from approximately 5 to approximately 30% (5%≤percentage by mass≤30%), relative to the total mass of said alkane mixture and the at least one C≥11 alkane is a branched C14 alkane present in said excipient at a percentage by mass from approximately 70 to approximately 95% (70%≤percentage by mass≤95%), relative to the total mass of said alkane mixture.

In one embodiment, the cosmetic excipient according to the invention is characterized in that said at least one C8-C10 alkane is a C10 alkane present in said excipient at a percentage by mass from approximately 10 to approximately 30% (10%≤percentage by mass≤30%), relative to the total mass of said alkane mixture and the at least one C≥11 alkane is a branched C14 alkane present in said excipient at a percentage by mass from approximately 70 to approximately 90% (70%≤percentage by mass≤90%), relative to the total mass of said alkane mixture.

In one embodiment, the cosmetic excipient according to the invention is characterized in that said at least one C8-C10 alkane is a C10 alkane present in said excipient at a percentage by mass from approximately 15 to approximately 30% (15%≤percentage by mass≤30%), relative to the total mass of said alkane mixture. and the at least one C≥11 alkane is a branched C14 alkane present in said excipient at a percentage by mass from approximately 70 to approximately 85% (70%≤percentage by mass≤85%), relative to the total mass of said alkane mixture.

In one embodiment, the cosmetic excipient according to the invention is characterized in that said at least one C8-C10 alkane is a C10 alkane, and the at least one C≥11 alkane is a linear C14 alkane.

In one embodiment, the cosmetic excipient according to the invention is characterized in that said at least one C8-C10 alkane is a C10 alkane, and the at least one C≥11 alkane is a branched C14 alkane.

The invention also relates to a cosmetic excipient comprising an alkane mixture as defined above, characterized in that said at least one C8-C10 alkane is a C10 alkane, and the at least one C≥11 alkane is a mixture of C12 and C14.

In one embodiment, the cosmetic excipient according to the invention is characterized in that said at least one C8-C10 alkane is a C10 alkane present in said excipient at a percentage by mass from approximately 1 to approximately 50% (10%≤percentage by mass≤50%), relative to the total mass of said alkane mixture, and the at least one C≥11 alkane is a branched C12 alkane present in said excipient at a percentage by mass from approximately 30 to approximately 80% (30%≤percentage by mass≤80%), relative to the total mass of said alkane mixture and the C14 alkane present in said excipient at a percentage by mass from approximately 30 to approximately 80% (30%≤percentage by mass≤80%), relative to the total mass of said alkane mixture.

In one embodiment, the cosmetic excipient according to the invention is characterized in that said at least one C8-C10 alkane is a C10 alkane, and the at least one C≥11 alkane is a mixture of a branched C12 alkane and a branched C14.

In one embodiment, the cosmetic excipient according to the invention is characterized in that said at least one C8-C10 alkane is a C10 alkane present in said excipient at a percentage by mass from approximately 10 to approximately 30% (10%≤percentage by mass≤30%), relative to the total mass of said alkane mixture. and the at least one C≥11 alkane is a branched C12 alkane present in said excipient at a percentage by mass from approximately 30% to approximately 70% (50%≤percentage by mass≤70%), relative to the total mass of said alkane mixture. and the C14 alkane present in said excipient at a percentage by mass from approximately 50 to approximately 70% (50%≤percentage by mass≤70%), relative to the total mass of said alkane mixture.

The invention also relates to a cosmetic excipient comprising an alkane mixture as defined above, characterized in that said at least one C8-C10 alkane is at least the n-decane present in said excipient at a percentage by mass from approximately 15 to approximately 25% (15%≤percentage by mass≤25%), relative to the total mass of said alkane mixture and the at least one C≥11 alkane is a mixture of n-dodecane present in said excipient at a percentage by mass from approximately 20 to approximately 60% (20%≤percentage by mass≤60%), relative to the total mass of said alkane mixture and the n-tetradecane present in said excipient at a percentage by mass from approximately 15 to approximately 45% (15%≤percentage by mass≤45%), relative to the total mass of said alkane mixture.

The invention also relates to a cosmetic excipient comprising an alkane mixture as defined above, characterized in that said at least one C8-C10 alkane is at least the n-decane present in said excipient at a percentage by mass of approximately 30% relative to the total mass of said alkane mixture, and the at least one C≥11 alkane is a mixture of n-dodecane present in said excipient at a percentage by mass of approximately 40% relative to the total mass of said alkane mixture and of n-tetradecane present in said excipient at a percentage by mass of approximately 30% relative to the total mass of said alkane mixture.

The invention also relates to a cosmetic excipient comprising an alkane mixture as defined above, characterized in that said at least one C8-C10 alkane is at least the n-decane present in said excipient at a percentage by mass from approximately 15 to approximately 5% (15%≤percentage by mass≤25%), relative to the total mass of said alkane mixture and the at least one C≥11 alkane is a mixture of n-dodecane present in said excipient at a percentage by mass from approximately 70 to approximately 80% (70%≤percentage by mass≤80%), relative to the total mass of said alkane mixture and the n-tetradecane present in said excipient at a percentage by mass from approximately 2 to approximately 10% (2%≤percentage by mass≤0%), relative to the total mass of said alkane mixture.

The invention also relates to a cosmetic excipient comprising an alkane mixture as defined above, characterized in that said at least one C8-C10 alkane is at least the n-decane present in said excipient at a percentage by mass from approximately 20% relative to the total mass of said alkane mixture, and the at least one C≥11 alkane is a mixture of n-dodecane present in said excipient at a percentage by mass of approximately 74% relative to the total mass of said alkane mixture and n-tetradecane present in said excipient at a percentage by mass of approximately 6% relative to the total mass of said alkane mixture

In one embodiment, said alkane mixture comprises:
30% n-decane;
40% n-dodecane; and
30% n-tetradecane.

In one embodiment, said alkane mixture comprises:
30% n-decane;
40% n-dodecane; and
30% n-tetradecane.

In one embodiment, said alkane mixture comprises:
10% n-decane;
85% n-dodecane; and
5% n-tetradecane.

In one embodiment, said alkane mixture comprises:
15% n-decane;
80% n-dodecane; and
5% n-tetradecane.

In one embodiment, said alkane mixture comprises:
20% n-decane;
75.6% n-dodecane; and
4.4% n-tetradecane.

In one embodiment, said alkane mixture comprises:
20% n-decane;
74% n-dodecane; and
6% n-tetradecane.

In one embodiment, said alkane mixture comprises:
20% n-decane;
70% n-dodecane; and
10% n-tetradecane.

In one embodiment, said alkane mixture comprises:
20% n-decane;
75% n-dodecane; and
5% n-tetradecane.

In one embodiment, said alkane mixture comprises:
30% n-decane; and
70% isohexadecan.

In one embodiment, said alkane mixture comprises:
5% n-decane;
30% isododecane; and
65% isoeicosane.

In one embodiment, said alkane mixture comprises:
5% n-decane;
25% isododecane; and
70% isoeicosane.

The invention also relates to a cosmetic excipient comprising an alkane mixture as defined above, characterized in that, it further contains a non-volatile silicone such as, for example, polysilicone 11.

The invention also relates to a cosmetic excipient comprising an alkane mixture as defined above, said excipient further comprising polysilicone 11, characterized in that said mixture of alkanes is present at a percentage by mass from approximately 65 to approximately 95% (65%≤percentage by mass≤95%), relative to the total mass of said excipient, and said polysilicone 11 is present at a percentage by mass from approximately 5 to approximately 35% (5%≤percentage by mass≤35%), relative to the total mass of said excipient.

The invention also relates to a cosmetic excipient comprising an alkane mixture as defined above, said excipient further comprising polysilicone 11, characterized in that said mixture of alkanes is present at a percentage by mass from approximately 75 to approximately 85% (75%≤percentage by mass≤85%), relative to the total mass of said excipient, and said polysilicone 11 is present at a percentage by mass from approximately 15 to approximately 25% (15%≤percentage by mass≤25%), relative to the total mass of said excipient.

The invention also relates to a cosmetic excipient comprising an alkane mixture as defined above, said excipient further comprising polysilicone 11, characterized in that said mixture of alkanes is present at a percentage by mass from approximately 80 to approximately 82% (80%≤percentage by mass≤82%), relative to the total mass of said excipient, and said polysilicone 11 is present at a percentage by mass from approximately 18 to approximately 20% (18%≤percentage by mass≤20%), relative to the total mass of said excipient.

The invention also relates to a cosmetic excipient comprising an alkane mixture as defined above, characterized in that said alkane mixture has a flash point less than or equal to 69° C. 69° C.).

The invention also relates to a cosmetic excipient comprising an alkane mixture as defined above, characterized in that said alkane mixture has a flash point between 60 and 69° C. (60° C. flash point 69° C.).

The invention also relates to a cosmetic excipient comprising an alkane mixture as defined above, characterized in that said alkane mixture has a flash point between 60 and 67° C. (60° C. flash point 67° C.).

The invention also relates to a cosmetic excipient comprising an alkane mixture as defined above, characterized in that said alkane mixture has a flash point between 62 and 65° C. (62° C. flash point 65° C.).

The invention also relates to a cosmetic excipient comprising an alkane mixture as defined above, characterized in that the percentage of evaporation at t=10 hours is between 10 and 30% (10%≤percentage of evaporation at t=10 h≤30%).

The invention also relates to a cosmetic excipient comprising an alkane mixture as defined above, characterized in that the percentage of evaporation at t=10 hours is between 12 and 20% (12%≤percentage of evaporation at t=10 h≤20%).

Cosmetic excipient according to the invention as defined above, characterized in that it is entirely obtained from raw materials of plant, bacterial or animal origin, preferably of plant origin.

In one embodiment, the alkanes according to the invention are obtained by dehydration of the corresponding Guerbet alcohol, followed by hydrogenation of the alkene obtained.

For example, 5-methylundecane will be obtained by dehydrating 2-butyloctanol-1 according to the reaction scheme below:

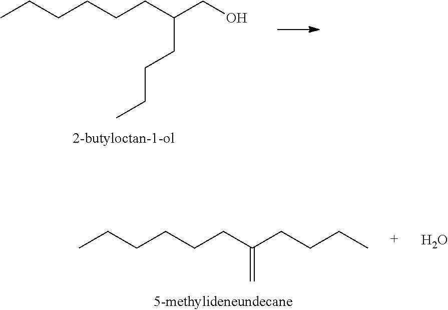

Then the alkene obtained is hydrogenated according to the reaction diagram below:

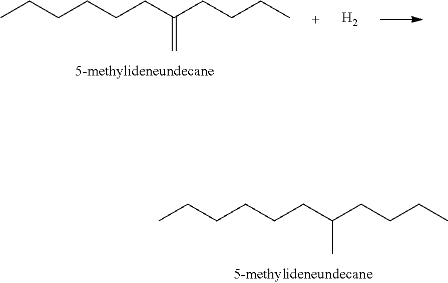

Guerbet alcohol will be obtained either by a classical Guerbet process through the condensation of two alcohols, or by a synthesis such as described in patent application US2012/0220806.

Use of a cosmetic excipient according to the invention as defined above, in the preparation of a cosmetic formulation of mascara type.

Use of a cosmetic excipient according to the invention as defined above, in the preparation of a biphasic makeup remover cosmetic formulation.

Use of a cosmetic excipient according to the invention as defined above, in the preparation of a cosmetic formulation for coloring the lips.

Use of a cosmetic excipient according to the invention as defined above, in the preparation of a cosmetic formulation of a hair rinse or non-rinse type.

Use of a cosmetic excipient according to the invention as defined above, in the preparation of a cosmetic formulation being an emulsion.

Use of a cosmetic excipient according to the invention as defined above, in the preparation of an oily phase.

Use of a cosmetic excipient according to the invention as defined above, in the preparation of an oily phase intended for an emulsion.

Use of a cosmetic excipient according to the invention as defined above, in substitution of at least one volatile silicone oil.

Use of a cosmetic excipient according to the invention as defined above, in substitution of at least one volatile silicone oil selected from the group comprising cyclotetrasiloxane (D4) (C8H23O4Si4, CAS 556-67-2), cyclopentasiloxane (D5) (C10H30O5Si5, CAS 541-02-6) and cyclohexasiloxane (D6) (C12H36O6Si6, CAS 540-97-6), and mixtures thereof.

Use of a cosmetic excipient according to the invention as defined above, in substitution of at least one volatile silicone oil being cyclotetrasiloxane (D4).

Use of a cosmetic excipient according to the invention as defined above, in substitution of at least one volatile silicone oil being cyclopentasiloxane (D5).

Use of a cosmetic excipient according to the invention as defined above, in substitution of at least one volatile silicone oil being cyclohexasiloxane (D6).

Use of a cosmetic excipient according to the invention as defined above, in substitution of at least one volatile silicone oil selected from the group comprising cyclotetrasiloxane (D4) (C8H23O4Si4, CAS 556-67-2), cyclopentasiloxane (D5) (C10H30O5Si5, CAS 541-02-6) and cyclohexasiloxane (D6) (C12H36O6Si6, CAS 540-97-6), and mixtures thereof characterized in that substitution is carried out while keeping the same mass quantity. This means that a certain mass of silicone oil is replaced by the same mass of a cosmetic excipient according to the invention.

Use of a cosmetic excipient according to the invention as defined above, in substitution of at least one volatile silicone oil being cyclotetrasiloxane (D4), characterized in that said substitution is carried out while keeping the same mass quantity.

Use of a cosmetic excipient according to the invention as defined above, in substitution of at least one volatile silicone oil being cyclopentasiloxane (D5), characterized in that said substitution is carried out while keeping the same mass quantity.

Use of a cosmetic excipient according to the invention as defined above, in substitution of at least one volatile silicone oil being cyclohexasiloxane (D6), characterized in that said substitution is carried out while keeping the same mass quantity.

Cosmetic formulation characterized in that it comprises, as a cosmetic excipient, a cosmetic excipient according to the invention as defined above.

The invention also relates to a cosmetic formulation characterized in that it comprises an alkane mixture, said mixture having a flash point less than or equal to 69° C. 69° C.).

Cosmetic formulation according to the invention as defined above, characterized in that it is selected from the group consisting of anhydrous cosmetic formulations, cosmetic formulations of the water-in-oil (W/O) type of emulsion, cosmetic formulations of the oil-in-water (O/W) type of emulsion, and cosmetic formulations in the form of multiple emulsions (in particular W/O, O/W or O/W/O), or even cosmetic formulations being dispersions.

Cosmetic formulation according to the invention as defined above, characterized in that it further comprises at least one of the constituents selected from the group comprising gelling agents or fatty phase structuring agents, in particular, waxes, gums, olefinic copolymers or even silicone elastomers, binders, in particular fatty acid soaps, aqueous phase gelling agents or thickeners such as acrylic or sulfonic homopolymers or copolymers, specifically AMPS-based, film-forming agents, such as PVP derivatives, acrylic latexes or silicone resins, dispersants such as fatty acid esters, W/0, 0/W or W/Si emulsifying agents, active ingredients, organic or inorganic photo-protective agents or UV filters, fillers, optionally treated hydrophobic and/or lipophobic pigments, organic or inorganic powders with a spherical and/or lamellar structure (such as silica, talc, mica, etc.), fibers of natural or synthetic origin, dyes, sequestering agents, pH adjusters, perfumes, preservatives, and mixtures thereof.

Cosmetic formulation according to the invention as defined above, characterized in that it is free from cyclic silicones (cyclomethicones), in particular of cyclotetrasiloxane and cyclopentasiloxane derivatives.

Cosmetic formulation according to the invention as defined above, characterized in that it is in a form selected from the group consisting of fluids, gels, creams, pastes, foams, pressed or cast compacts and solid products, for example in stick form.

Cosmetic formulation according to the invention as defined above, characterized in that it is used as a formulation for face and/or body care or hygiene, a moisturizing formulation, an anti-aging formulation (anti-wrinkle and/or firming), a depigmenting formulation, a pro-pigmenting formulation, a self-tanning formulation, a slimming formulation, a deodorant formulation, a antiperspirant formulation, a UV protection formulation, a cleansing formulation, a formulation to remove makeup, a biphasic formulation for makeup removal, a bath formulation, a hair formulation (in particular, a shampoo, conditioner, styling product, in particular, a straightening product), a formulation for coloring hair, a makeup formulation, (in particular a foundation, a lipstick, a lip gloss, a formulation for coloring the lips, a blusher or eye shadow, a mascara, an eyeliner or even a nail polish).

Cosmetic formulation according to the invention as defined above, characterized in that at least said at least one C≥11 alkane is branched, and in that said cosmetic formulation is selected from the group of cosmetic formulations of water-in-oil (W/O) emulsion type, cosmetic formulations of the oil-in-water (O/W) emulsion type, and cosmetic formulations in the form of multiple emulsions (in particular W/O, O/W or O/W/O).

Cosmetic formulation according to the invention as defined above, characterized in that said at least one C≥11 alkane is at least a branched C12, and in that said cosmetic formulation is selected from the group of cosmetic formulations of water-in-oil (W/O) emulsion type, cosmetic formulations of the oil-in-water (O/W) type of emulsion, and cosmetic formulations in the form of multiple emulsions (in particular W/O, O/W or O/W/O).

Cosmetic formulation according to the invention as defined above, characterized in that said at least one C≥11 alkane is at least a branched C14, and in that said cosmetic formulation is selected from the group of cosmetic formulations of water-in-oil (W/O) emulsion type, cosmetic formulations of the oil-in-water (O/W) type of emulsion, and cosmetic formulations in the form of multiple emulsions (in particular W/O, O/W or O/W/O).

Cosmetic formulation according to the invention as defined above, characterized in that said at least one C≥11 alkane is at least a mixture of a branched C12 and a branched C14, and in that said cosmetic formulation is selected from the group of cosmetic formulations of water-in-oil (W/O) emulsion type, cosmetic formulations of the oil-in-water (O/W) type of emulsion, and cosmetic formulations in the form of multiple emulsions (in particular W/O, O/W or O/W/O).

### EXAMPLES

### Part A—Examples of Cosmetic Compositions

### Example A1: Formulations According to the Invention (Emulsions/Spray Lotions)

The following formulations according to the invention are prepared according to techniques well known to those skilled in the art, such as the techniques described in Conception des cosmétiques, La formulation, Anne Marie Pensé-Lhéritier, July 2014, Lavoisier:

| | | | |
|---|---|---|---|
| | Commercial | | Percentage by mass |
| Phase | Name | INCI | (%) |

Formulation 1 (emulsion/lotion spray)

| | | | |
|---|---|---|---|
| A | Water | Aqua | Qsp 100 |
| B | Isolan PDI | Diisostearoyl Polyglyceryl-3 | 2.5 |
| | (EVONIK) | Dimer Dilinoleate | |
| | Pongamia | Pongamina glabra seed oil | 10 |
| | Butter 80 Plus | (and) C18-21 alkane | |
| | (BIOSYNTHIS) | | |
| | UV Cut TiO2- | Titanium dioxide (and) | 20 |
| | 60-VL | Coconut Alkanes (and) | |
| | (GRANT | Stearic acid (and) | |
| | INDUSTRIES | Polyhdroxystearic acid (and) | |
| | INC.) | Alumina (and) | |
| | | Coco-caprylate/caprate | |
| | Mixture of | Tetradecane (and) Decane | 25 |
| | n-decane (30%) | | |
| | and | | |
| | n-tetradecane | | |
| | (70%) | | |
| C | MinaSolve | Pentylene Glycol (and) | 2 |
| | Green C | Glyceryl Caprylate/Caprate | |
| | (MINASOLVE) | | |

Formulation 2 (emulsion/lotion spray)

| | | | |
|---|---|---|---|
| A | Water | Aqua | Qsp 100 |
| B | Isolan PDI | Diisostearoyl | 2.5 |
| | (EVONIK) | Polyglyceryl- | |
| | | 3 Dimer Dilinoleate | |
| | Pongamia Butter | Pongamina glabra seed | 10 |
| | 80 Plus | oil | |
| | (BIOSYNTHIS) | (and) C18-21 alkane | |
| | UV Cut TiO2-60- | Titanium dioxide (and) | 20 |
| | VL | Coconut Alkanes(and) | |
| | (GRANT | Stearic acid (and) | |
| | INDUSTRIES | Polyhdroxystearic acid | |
| | INC.) | (and) Alumina (and) | |
| | | Coco-caprylate/caprate | |
| | Mixture of | Isohexadecane (and) | 25 |
| | n-decane (30%) | Decane | |
| | and | | |
| | n-isohexadecane | | |
| | (70%) | | |
| C | MinaSolve Green | Pentylene Glycol (and) | 2 |
| | C (MINASOLVE) | Glyceryl | |
| | | Caprylate/Caprate | |

Formulations 1 and 2 are prepared as follows:
heating of phases A and B (60° C.);
emulsification of phase A with phase B (stirring);
addition of phase C while continuing to stir.

Stability is measured by the technique of visual analysis. A sample of each of the emulsions is placed in a transparent container and observed with the naked eye at regular time intervals, as recommended in technical report TR 13097: Guidelines for the characterization of dispersion stability.

Formulation 2 is very stable (no phase shift after three weeks), whereas formulation 1 is phase shifted after one day. The particular advantage of including branched C≥11 alkanes is therefore demonstrated, in the context of the use of a cosmetic excipient according to the invention in a formulation of the emulsion type.

### Example A2: Formulations According to the Invention (Mascara)

The following formulations according to the invention are prepared:

| | | | |
|---|---|---|---|
| | | | Percentage |
| Phase | Commercial Name | INCI | by mass (%) |

Formulation 3 (emulsion/mascara)

| | | | |
|---|---|---|---|
| A | Water | Aqua | 18.00 |
| | NaCl | Sodium Chloride | 0.70 |
| B | Natural Beewax | Cera alba | 6.00 |
| | Sunflower Wax | Helianthus annuus | 4.00 |
| | | seed | |
| | | Wax | |
| | Mixture of n-decane | Decane (and) | Qsp 100.00 |
| | (50%) and | Isohexadecane | |
| | n-isohexadecane | | |
| | (50%) | | |
| | Xiameter ™ RSN- | Cyclopentasiloxane | 5.00 |
| | 0749 Resin | (and) | |
| | (DOW CORNING) | Trimethyisiioxysilicate | |
| | Nikkomulese WO | Cyclopentasiloxane | 16.00 |
| | (NIKKOL) | (and) | |
| | | PEG-10 Dimethicone | |
| | | (and) Disteardimonium | |
| | | Hectorite | |
| C | SA C335000-10 | CI 77499, Dimethicone | 20.00 |
| | (MIYOSHI GROUP) | | |
| | Micropearl M305 | Methylmethacrylate | 3.00 |
| | (SEPPIC) | rosspolymer | |
| | Methylmethacrylate | | |
| | crosspolymer 3.00 | | |

Formulation 4 (emulsion/mascara)

| | | | |
|---|---|---|---|
| A | Water | Aqua | 18.00 |
| | NaCl | Sodium Chloride | 0.70 |
| B | Natural Beewax | Cera Alba | 6.00 |
| | Sunflower Wax | Helianthus annuus | 4.00 |
| | | seed | |
| | | wax | |
| | Mixture of n-decane | Decane(and) | Qsp 100.00 |
| | (50%) and | Isohexadecane | |
| | n-methylundecane | | |
| | (50%) | | |
| | Xiameter ™ RSN- | Cyclopentasiloxane | |
| | 0749 Resin (DOW | (and) | 5.00 |
| | CORNING) | Trimethylsiloxysilicate | |
| | Nikkomulese WO | Cyclopentasiloxane | 16.00 |
| | (NIKKOL) | (and) | |
| | | PEG-10 Dimethicone | |
| | | (and) | |
| | | Disteardimonium | |
| | | Hectorite | |
| C | SA C335000-10 | CI 77499, Dimethicone | 20.00 |
| | (MIYOSHI GROUP) | | |
| | Micropearl M305 | Methylmethacrylate | 3.00 |
| | (SEPPIC) | crosspolymer | |
| | Methylmethacrylate | | |
| | crosspolymer 3.00 | | |

Formulations 3 and 4 are prepared as follows:
preparation of Phase A and heating (75° C.);
preparation of Phase B and heating (85° C.);
mixture of Phases A and B;
addition of Phase C to Phase B with vigorous stirring;
bring the mixture to 80° C., and the addition of Phase A to Phases B+C;
mix for 15 minutes;
cooling to 25° C.

### Example A3: Formulations According to the Invention (Biphasic Formulation for Makeup Removal)

The following formulation according to the invention is prepared:

Formulation 5 (biphasic formulation for makeup removal)

| | | | Percentage by mass |
|---|---|---|---|
| Phase | Commercial Name | INCI | (%) |
| A (Oily | Mixture of n-decane | Dodecane (and) | 99.60 |
| phase) | (30%) and | Decane (and) | |
| | n-dodecane (40%) | Tetradecane | |
| | and n-tetradecane | | |
| | (30%) | | |
| | 0.5% CI 60725 | Squalane (and) CI | 0.20 |
| | solution in | 60725 | |
| | SQUALANE | | |
| | 0.5% CI 61565 | Squalane (and) CI | 0.20 |
| | solution in | 61565 | |
| | SQUALANE | | |
| B (water | Water | Aqua | 85.70 |
| phase) | K2HPO4 | Dipotassium | 0.77 |
| | | Phosphate | |
| | Citric acid | Citric Acid | 0.48 |
| | Glycerine | Glycerine | 5.00 |
| | Sodium Chloride | Sodium Chloride | 0.05 |
| | Hexylene glycol | Hexylene Glycol | 2.00 |
| | 1,3-propanediol | propanediol | 5.00 |
| | 0.5% CI 42090 | Aqua (and) CI 42090 | 1.00 |
| | aqueous solution | | |

It should be noted that for this formulation 5, the percentage by mass given are relative to the total mass of the phase concerned.

In said formulation 5, the aqueous phase/oily phase proportions are 50/50.

### Example A4: Formulation According to the Invention (Formulation for Coloring the Lips)

The following formulation according to the invention is prepared:

Formulation 6 (formulation for coloring the lips)

| | | | Percentage |
|---|---|---|---|
| | | | by |
| Phase | Commercial Name | INCI | mass (%) |
| A | ViscoplastGreen 3000 | Dili noleicacid/ | QSP |
| | (BIOSYNTHIS) | Propanediolcopolymer | 100.00 |
| | VegelightSilk and | Decane (and) Tetradecane | 6.00 |
| | Polypropyl- | (and) | |
| | silsesquioxane | Polypropylsilsesquioxane | |
| | Biosynth DIM-18 | Diisostearylmalate | 5.00 |
| | Argan Oil | Argania spinosa (Argan) | 3.00 |
| | | seed oil | |
| | Jojoba Oil | Simmondsia chinensis oil | 3.00 |
| | Tinogard TT | Pentaerythrityl | 0.05 |
| | | tetra-di-t- | |
| | | butylhydroxyhydrocinnamate | |
| | Broyat DC Red 7 at | Diisostearylmalate | 25.00 |
| | 1% dans Biosynth | (and) CI15850 | |
| | DIM-18 | | |

### Example A5: Formulation According to the Invention (High Quality Lip Gloss)

Formulation 7 (high quality lip gloss)

| | | | Percentage by |
|---|---|---|---|
| Phase | Commercial Name | INCI | mass (%) |
| A | Candelilla Wax | Candelilla cera | 6.16 |
| | Sunflower Seed Oil | Helianthus annuus seed | 5.00 |
| | | oil | |
| | Sweet Almond Oil | Prunus amygdalus | 4.00 |
| | | dulcis Oil | |
| | Vegelight Silk | Coconut Alkanes | 50.00 |
| | 21% Decane | | |
| | 74% Dodecane | | |
| | 5% Tetradecane | | |
| | Viscoplast Green 700 | C18-21 alkane (and) | 20.00 |
| | HVL | Dilinoleic | |
| | | acid/Propanediol | |
| | | Copolymer (and) | |
| | | Coco-caprylate/caprate | |
| | Mixture of SKYLINE F | C10-18 | 1.70 |
| | and FDC YELLOW 6 | TRIGLYCERIDES, | |
| | Al-I2 (50%/50%) | POLYISOPRENE, | |
| | | ACETYLATED | |
| | | GLYCOL STEARATE, | |
| | | GLYCINE SOJA OIL, | |
| | | LECITHIN | |
| | | CI 15985:1, | |
| | | SOPROPYL TITANIUM | |
| | | TRIISOSTEARATE | |
| | Mixture of SKYLINE F | C10-18 | 3.64 |
| | and SA-TAO77891- | TRIGLYCERIDES, | |
| | 10 (50%/50%) | POLYISOPRENE, | |
| | | ACETYLATED | |
| | | GLYCOL | |
| | | STEARATE, | |
| | | GLYCINE | |
| | | SOJA OIL, LECITHIN | |
| | | CI 77891, | |
| | | DIMETHICONE | |
| | Mixture of SKYLINE F | C10-18 | 2.70 |
| | and SA-C33128-10 | TRIGLYCERIDES, | |
| | (50%/50%) | POLYISOPRENE, | |
| | | ACETYLATED | |
| | | GLYCOL | |
| | | STEARATE, | |
| | | GLYCINE SOJA OIL, | |
| | | LECITHIN | |
| | | CI 77491, | |
| | | DIMETHICONE | |
| | Mixture of SKYLINE F | C10-18 | 3.05 |
| | (60%) and DC RED 27 | TRIGLYCERIDES, | |
| | Al-I2 (40%) | POLYISOPRENE, | |
| | | ACETYLATED | |
| | | GLYCOL STEARATE, | |
| | | GLYCINE SOJA OIL, | |
| | | LECITHIN | |
| | | CI 45410:2, | |
| | | SOPROPYL | |
| | | TITANIUM | |
| | | TRIISOSTEARATE | |
| | Mixture of SKYLINE F | C10-18 | 3.75 |
| | (60%) and DC RED 7 | TRIGLYCERIDES, | |
| | Ca-C-TTB2 (40%) | POLYISOPRENE, | |
| | | ACETYLATED | |
| | | GLYCOL STEARATE, | |
| | | GLYCINE SOJA OIL, | |
| | | LECITHIN | |
| | | CI 15850:1, | |
| | | SOPROPYL | |
| | | TITANIUM | |
| | | TRIISOSTEARATE | |

### Example A6: Comparison of Serum, Hair Formulations

The following hair serum formulations were compared for their properties when applied to the hair.

| | | | Hair Serum Formulations | | |
|---|---|---|---|---|---|
| | | | Reference | Formulation | Formulation |
| | | | Formulation | 1 | 2 |
| | | | Percentage | Percentage | Percentage |
| | | | by mass | by mass | by mass |
| Phase | Commercial Name | INCI | (%) | (%) | (%) |
| A | Xiameter PMX-1501 | Cyclopentasiloxane | 30 | 30 | — |
| | Fluid (DOW | (and) Dimethiconol | | | |
| | CORNING) | | | | |
| | Xiameter PMX-0245 | Cyclopentasiloxane | 68 | — | — |
| | (DOW CORNING) | | | | |
| | 20% n-decane | Coconut Alkanes | — | 68 | 30 |
| | 34% n-dodecane | (and) Dimethiconol | | | |
| | 15% n-hexadecane | | | | |
| | 15% n-octadecane | | | | |
| | 16% dimethiconol | | | | |
| | 35% n-decane | Coconut Alkanes | — | — | 68 |
| | 5% n-octadecane | | | | |
| | 60% 9-methyl | | | | |
| | nonadecane | | | | |
| B | Cold Pressed | Argania spinosa | 1 | 1 | 1 |
| | Argan Oil | kernel oil | | | |
| | KaranSun Oil | Pongamia glabra | 0.5 | 0.5 | 0.5 |
| | (BIOSYNTHIS) | seed oil | | | |
| | Perfume | Perfume | 0.5 | 0.5 | 0.5 |

| | | | |
|---|---|---|---|
| Total silicone content | 98% | 30% | 4.50% |

The hair serum formulations were compared as follows:

3 locks of hair of approximately 4 g and with a length of approximately 17 cm are washed vigorously with 25 ml of Texapon NSO solution at 12% and are rinsed and then dried.

0.2 g of the formulations described in the table above are applied to each of the locks in order to compare said formulations.
The lock of hair treated with Formulation 2 exhibits better qualities than the reference formulation, in particular with regard to ease of styling; however, the lock of hair treated with Formulation 1 is not suitable for very fine hair.

### Example A7: Hair Balm

Reference Balm

| Commercial Name | INCI Name | % |
|---|---|---|
| Xiameter ™ PMX-1501 | Cyclopentasiloxane | 25.00 |
| Fluid | (and) Dimethiconol | |
| Belsil ® DM 60000 | Dimethicone | 3.00 |
| Marcol 82 | Paraffinum liquidum | 25.00 |
| DOW CORNING ® 245 | Cyclopentasiloxane | 46.00 |
| Perfume | Perfume | 1.00 |

### Example A7a Balm

| Commercial Name | INCI Name | % |
|---|---|---|
| Xiameter ™ PMX-1501 | Cyclopentasiloxane | 25.00 |
| Fluid | (and) Dimethiconol | |
| Belsil ® DM 60000 | Dimethicone | 3.00 |
| Marcol 82 | Paraffinum liquidum | 25.00 |
| 30% Decane | Coconut alkanes | 46.00 |
| 40% Dodecane | | |
| 30% Tetradecane | | |
| Perfume | Perfume | 1.00 |

### Example A7b Balm

| Commercial Name | INCI Name | % |
|---|---|---|
| Xiameter ™ PMX-1501 | Cyclopentasiloxane | 25.00 |
| Fluid | (and) Dimethiconol | |
| Belsil ® DM 60000 | Dimethicone | 3.00 |
| Marcol 82 | Paraffinum liquidum | 25.00 |
| 35% Decane | Coconut alkanes | 46.00 |
| 5% Octadecane | | |
| 60% 9-methyl | | |
| Nonadecane | | |
| Perfume | Perfume | 1.00 |

The A7a and A7b balms provide light and creamy touches to the hands and silky touches to the hair.

Hair is shiny and easy to style.

Part B—Physicochemical Characteristics of the Excipients According to the invention

### Example B1: Evaporation Characteristics of an Excipient Comprising an Alkane Mixture According to the Invention

An alkane mixture comprising 30% n-decane, 40% n-dodecane and 30% n-tetradecane was investigated for its evaporation characteristics.

The evaporation conditions are the following: LABOMODERNE THERMOBALANCE KERN DBS 60-3 desiccator, 20° C., under controlled atmosphere.

The evaporation curve obtained is given below:

The measurements of the evaporation rates of the mixture comprising 30% n-decane, 40% n-dodecane and 30% n-tetradecane are given below in comparison with those of a mixture according to the prior art comprising 70% n-dodecane, and 30% n-tetradecane and those of cyclopentasiloxane.

| | | |
|---|---|---|
| Time | Total | Residual |
| (min) | Mass (g) | Mass (%) |

Evaporation of an excipient according
to the invention (n-decane (30%), n-
dodecane (40%) and n-tetradecane (30%))

| | | |
|---|---|---|
| 0 | 5.32 | 100 |
| 7 | 5.31 | 99.68 |
| 20 | 5.30 | 99.23 |
| 39 | 5.28 | 98.61 |
| 59 | 5.27 | 98.01 |
| 79 | 5.25 | 97.36 |
| 104 | 5.23 | 96.70 |
| 129 | 5.21 | 96.05 |
| 159 | 5.19 | 95.33 |
| 189 | 5.17 | 94.62 |
| 219 | 5.15 | 94.04 |
| 249 | 5.13 | 93.28 |
| 279 | 5.10 | 92.55 |
| 309 | 5.09 | 92.00 |
| 358 | 5.05 | 90.54 |
| 429 | 5.01 | 89.39 |
| 479 | 4.98 | 88.23 |
| 1438 | 4.42 | 69.27 |

Evaporation of a comparative alkane
mixture (n-dodecane (70%) and
n-tetradecane (30%))

| | | |
|---|---|---|
| 0 | 5.38 | 100.00 |
| 20 | 5.38 | 99.83 |
| 50 | 5.37 | 99.51 |
| 90 | 5.36 | 99.13 |
| 130 | 5.34 | 98.78 |
| 170 | 5.33 | 98.40 |
| 210 | 5.32 | 98.00 |
| 250 | 5.31 | 97.61 |
| 300 | 5.30 | 97.21 |
| 356 | 5.28 | 96.65 |
| 1620 | 5.00 | 87.14 |

Cyclopentasiloxane (D5) Evaporation

| | | |
|---|---|---|
| 0 | 5.39 | 100 |
| 7 | 5.38 | 99.82 |
| 20 | 5.37 | 99.55 |
| 39 | 5.36 | 99.16 |
| 59 | 5.35 | 98.75 |
| 79 | 5.33 | 98.31 |
| 104 | 5.32 | 97.82 |
| 129 | 5.31 | 97.36 |
| 159 | 5.29 | 96.85 |
| 189 | 5.28 | 96.44 |
| 219 | 5.26 | 95.94 |
| 249 | 5.25 | 95.51 |
| 279 | 5.24 | 95.12 |
| 309 | 5.23 | 94.55 |
| 356 | 5.20 | 93.72 |
| 429 | 5.17 | 92.74 |
| 479 | 5.14 | 91.97 |
| 1436 | 4.54 | 71.85 |

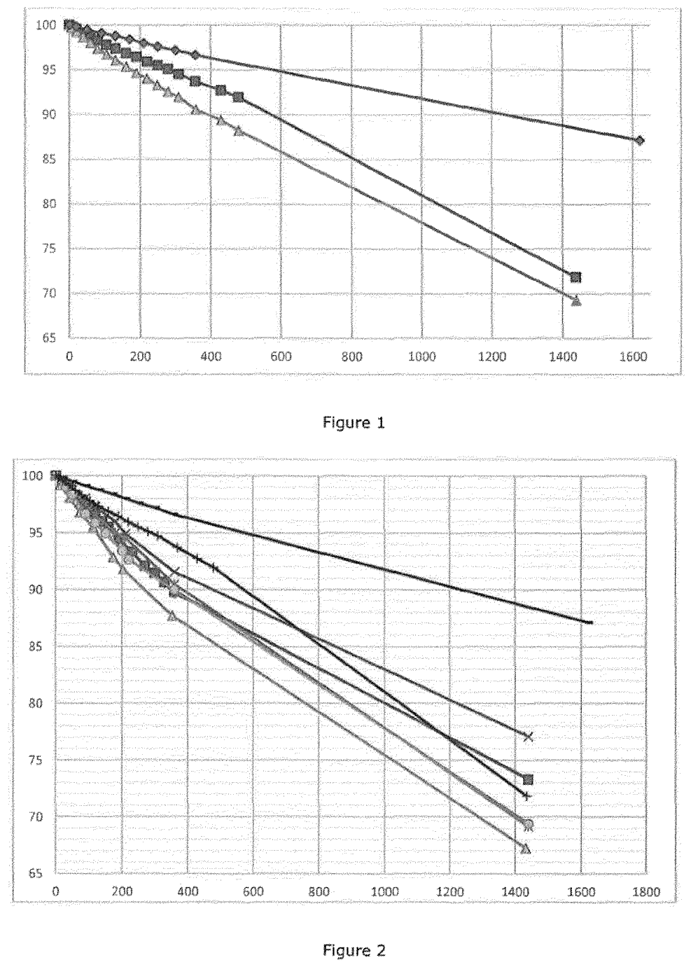

The results are shown in FIG. 1, the curve represented by diamond-shaped is the evaporation curve of a mixture according to the prior art comprising 70% n-dodecane, and 30% n-tetradecane, the curve represented by squares is the evaporation curve of cyclopentasiloxane and the curve represented by the diamond-shapes is the evaporation curve of a mixture according to the invention comprising 30% n-decane, 40% n-dodecane and 30% n-tetradecane.

It is observed that the curve of the mixture according to the invention is perfectly aligned with that of cyclopentasiloxane.

In addition, it has been demonstrated that the evaporation is particularly surprising and not deducible from that of each of the alkanes taken separately.

### Example B2: Evaporation Characteristics of an Excipient Comprising an Alkane Mixture According to the Invention

Several other excipients according to the invention (alkane mixtures) were tested with regard to their rate of evaporation, under conditions identical to those described above.

Mixtures of n-decane/n-dodecane/n-tetradecane

An mixture of alkanes consisting of 10% n-decane, 85% n-dodecane and 5% n-tetradecane was tested. The results are presented in the table below:

Evaporation of an excipient according
to the invention (10% n-decane, 85%
n-dodecane and 5% n-tetradecane)

| Time | Total | Residual |
|---|---|---|
| (min) | Mass (g) | Mass (%) |
| 0 | 5.52 | 100 |
| 20 | 5.51 | 99.53 |
| 50 | 5.49 | 98.98 |
| 90 | 5.46 | 98.03 |
| 120 | 5.44 | 97.29 |
| 180 | 5.39 | 95.98 |
| 210 | 5.36 | 94.90 |
| 360 | 5.26 | 91.58 |
| 1440 | 4.81 | 77.10 |

An mixture of alkanes consisting of 15% n-decane, 80% n-dodecane and 5% tetradecane was tested. The results are presented in the table below:

Evaporation of an excipient according
to the invention (15% n-decane, 80%
n-dodecane and 5% tetradecane)

| Time | Total | Residual |
|---|---|---|
| (min) | Mass (g) | Mass (%) |
| 0 | 5.46 | 100 |
| 20 | 5.44 | 99.43 |
| 50 | 5.42 | 98.74 |
| 90 | 5.39 | 97.58 |
| 120 | 5.36 | 96.63 |
| 180 | 5.31 | 95.02 |
| 210 | 5.29 | 94.52 |
| 360 | 5.17 | 90.51 |
| 1440 | 4.52 | 69.14 |

A alkane mixture consisting of 20% n-decane, 75.6% n-dodecane and 4.4% tetradecane was tested. The results are presented in the table below:

Evaporation of an excipient according
to the invention (20% n-decane, 75.6%
n-dodecane and 4.4% tetradecane)

| Time | Total | Residual |
|---|---|---|
| (min) | Mass (g) | Mass (%) |
| 0 | 5.43 | 100 |
| 30 | 5.40 | 99.04 |
| 50 | 5.38 | 98.33 |
| 90 | 5.33 | 96.66 |
| 120 | 5.31 | 95.87 |
| 150 | 5.28 | 94.95 |
| 200 | 5.23 | 93.40 |
| 220 | 5.21 | 92.63 |
| 360 | 5.13 | 89.99 |
| 1440 | 4.51 | 69.42 |

An mixture of alkanes consisting of 20% n-decane, 74% n-dodecane and 6% tetradecane was tested. The results are presented in the table below:

Evaporation of an excipient according
to the invention (20% n-decane, 74%
n-dodecane and 6% tetradecane)

| Time | Total | Residual |
|---|---|---|
| (min) | Mass (g) | Mass (%) |
| 0 | 5.37 | 100 |
| 20 | 5.36 | 99.49 |
| 40 | 5.34 | 98.93 |
| 60 | 5.32 | 98.25 |
| 80 | 5.31 | 97.79 |
| 100 | 5.29 | 97.23 |
| 120 | 5.27 | 96.64 |
| 140 | 5.25 | 95.96 |
| 160 | 5.24 | 95.44 |
| 190 | 5.21 | 94.39 |
| 230 | 5.17 | 93.24 |
| 270 | 5.14 | 92.06 |
| 300 | 5.12 | 91.43 |
| 330 | 5.09 | 90.58 |
| 360 | 5.07 | 89.72 |
| 1440 | 4.57 | 73.36 |

A alkane mixture consisting of 20% n-decane, 70% n-dodecane and 10% tetradecane was tested. The results are presented in the table below:

Evaporation of an excipient according
to the invention (20% n-decane, 70%
n-dodecane and 10% tetradecane)

| Time | Total | Residual |
|---|---|---|
| (min) | Mass (g) | Mass (%) |
| 0 | 5.38 | 100 |
| 20 | 5.36 | 99.51 |
| 40 | 5.35 | 98.94 |
| 60 | 5.33 | 98.29 |
| 80 | 5.31 | 97.83 |
| 100 | 5.30 | 97.25 |
| 120 | 5.28 | 96.68 |
| 140 | 5.26 | 96.02 |
| 160 | 5.28 | 95.50 |
| 190 | 5.21 | 94.46 |
| 230 | 5.18 | 93.31 |
| 270 | 5.14 | 92.11 |
| 300 | 5.12 | 91.50 |
| 330 | 5.10 | 90.67 |
| 360 | 5.07 | 89.81 |
| 1440 | 4.58 | 73.31 |

An mixture of alkanes consisting of 20% n-decane, 75% n-dodecane and 5% tetradecane was tested. The results are presented in the table below:

Evaporation of an excipient according
to the invention (20% n-decane, 75%
n-dodecane and 5% tetradecane)

| Time | Total | Residual |
|---|---|---|
| (min) | Mass (g) | Mass (%) |
| 0 | 5.45 | 100 |
| 13 | 5.43 | 99.17 |
| 43 | 5.39 | 98.07 |
| 73 | 5.36 | 96.78 |
| 113 | 5.32 | 95.45 |
| 173 | 5.24 | 92.86 |
| 203 | 5.20 | 91.77 |
| 353 | 5.08 | 87.70 |
| 1433 | 4.46 | 67.23 |

FIG. 2 represents the percentage of non-evaporated material (expressed in %) as a function of time (expressed in seconds) of a certain number of compound (cyclopentasiloxane) and alkane mixtures. The curve represented by “−” (the curve on the graph which shows the mixture having the least tendency to evaporate) is the evaporation curve of a alkane mixtures according to the prior art comprising 70% n-dodecane and 30% n-tetradecane.

The curve represented by “+” is the evaporation curve of cyclopentasiloxane.

The curve represented by the diamond-shapes is the evaporation curve of a mixture according to the invention comprising 20% of n-decane, 74% of n-dodecane and 6% of n-tetradecane.

The curve represented by the squares is the evaporation curve of a mixture according to the invention comprising 20% of n-decane, 70% n-dodecane and 10% of n-tetradecane.

The curve represented by the triangles is the evaporation curve of a mixture according to the invention comprising 20% of n-decane, 75% of n-dodecane and 5% of n-tetradecane.

The curve represented by the “x” is the evaporation curve of a mixture according to the invention comprising 10% n-decane, 85% n-dodecane and 5% n-tetradecane.

The curve represented by the stars is the evaporation curve of a mixture according to the invention comprising 15% n-decane, 80% n-dodecane and 5% n-tetradecane.

The curve represented by the circles is the evaporation curve of a mixture according to the invention comprising 20% n-decane, 70% n-dodecane and 10% n-tetradecane.

It is noted that the mixtures according to the invention have an evaporation profile closer to cyclopentasiloxane than the alkane mixtures according to the prior art.

N-Decane/Isohexadecane Mixture

An alkane mixture consisting of 30% n-decane and 70% isohexadecane was tested. The results are presented in the table below:

Evaporation of an excipient according
to the invention (30% n-decane
and 70% isohexadecane)

| Time | Total | Residual |
|---|---|---|
| (min) | Mass (g) | Mass (%) |
| 0 | 5.41 | 100 |
| 20 | 5.38 | 99.15 |
| 50 | 5.33 | 97.46 |
| 90 | 5.26 | 95.15 |
| 130 | 5.20 | 93.13 |
| 170 | 5.14 | 91.07 |
| 210 | 5.07 | 88.86 |
| 250 | 5.01 | 86.92 |
| 300 | 4.96 | 85.05 |
| 360 | 4.89 | 82.74 |
| 1620 | 4.27 | 62.38 |

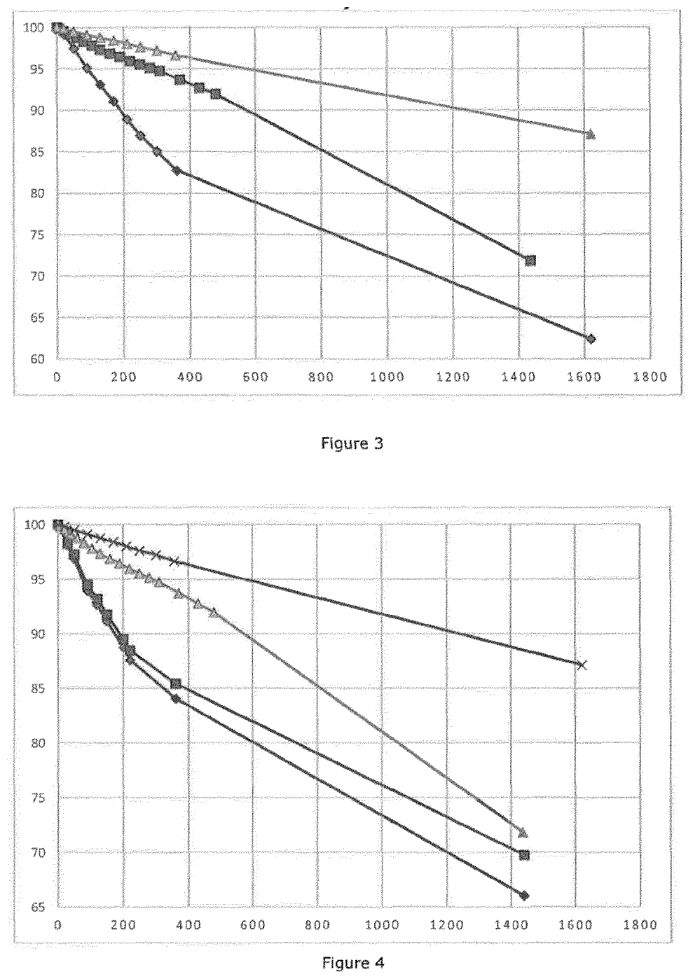

FIG. 3 represents the percentage of non-evaporated material (expressed in %) as a function of time (expressed in seconds) of a certain number of compound (cyclopentasiloxane) and alkane mixtures.

The curve represented by triangles (the curve on the graph which shows the mixture having the least tendency to evaporate) is the evaporation curve of an alkane mixture according to the prior art comprising 70% n-dodecane and 30% n-tetradecane.

The curve represented by the squares is the evaporation curve of cyclopentasiloxane.

The curve represented by the diamond-shapes is the evaporation curve of a mixture according to the invention comprising 30% n-decane, 70% n-dodecane.

It is noted that the mixtures according to the invention have an evaporation profile closer to cyclopentasiloxane than the alkane mixtures according to the prior art, from approximately T=900 seconds.

It is also notable that despite its large carbon number (C16), isohexadecane makes it possible to obtain a very volatile excipient (for example, more volatile than the same formulation comprising, instead of isohexadecane, a mixture of n-dodecane and n-tetradecane, see examples presented above).

Viscosity measurements at 40° C. were carried out on the following compounds or mixture:
cyclopentasiloxane: 3.04 mm²/s;
isohexadecane: 3.18 mm²/s; and
n-decane (30%)/isohexadecane (70%) mixture: 1.89 mm²/s.

It is notable that the use of isohexadecane makes it possible to obtain a viscosity close to that of cyclopentasiloxane.

N-Decane/Isododecane/Isoeicosane Mixtures

A mixture of alkanes consisting of 5% n-decane, 30% isododecane and 65% isoeicosane (C20) was tested. The results are presented in the table below:

Evaporation of an excipient according to the
invention (5% n-decane, 30% n-isododecane
and 65% isoeicosane)

| Time | Total | Residual |
|---|---|---|
| (min) | Mass (g) | Mass (%) |
| 0 | 5.44 | 100 |
| 30 | 5.38 | 98.13 |
| 50 | 5.35 | 96.90 |
| 90 | 5.26 | 93.94 |
| 120 | 5.22 | 92.64 |
| 150 | 5.17 | 91.16 |
| 200 | 5.10 | 88.76 |
| 220 | 5.06 | 87.57 |
| 360 | 4.96 | 84.07 |
| 1440 | 4.41 | 66.02 |

A mixture of alkanes consisting of 5% n-decane, 25% isododecane and 70% isoeicosane (C20) was tested. The results are presented in the table below:

Evaporation of an excipient according to the
invention (5% n-decane, 25% isododecane
and 70% isoeicosane)

| Time | Total | Residual |
|---|---|---|
| (min) | Mass (g) | Mass (%) |
| 0 | 5.42 | 100 |
| 30 | 5.37 | 98.27 |
| 50 | 5.34 | 97.27 |
| 90 | 5.25 | 94.47 |
| 120 | 5.21 | 93.22 |
| 150 | 5.17 | 91.75 |
| 200 | 5.10 | 89.48 |
| 220 | 5.07 | 88.47 |
| 360 | 4.98 | 85.43 |
| 1440 | 4.50 | 69.76 |

FIG. 4 represents the percentage of non-evaporated material (expressed in %) as a function of time (expressed in seconds) of a certain number of compound (cyclopentasiloxane) and alkane mixtures.

In FIG. 4, the curve represented by the “x” is the evaporation curve of a mixture according to the prior art comprising 70% n-dodecane and 30% n-tetradecane.

The curve represented by the triangles is the evaporation curve of cyclopentasiloxane.

The curve represented by the diamond-shapes is the evaporation curve of a mixture according to the invention comprising 5% n-decane, 30% isododecane and 65% isoeicosane.

The curve represented by the squares is the evaporation curve of a mixture according to the invention comprising 5% n-decane, 25% isododecane and 70% isoeicosane.

It is noted that the mixtures according to the invention have an evaporation profile closer to cyclopentasiloxane than the alkane mixtures according to the prior art, from approximately T=800 seconds.

Part C'Synthesis of the Alkanes Constituting the Excipients According to the Invention

### Example C1—Synthesis of 2,3,6, trimethylheptane

Guerbet synthesis of a C10 alkane from isoamyl alcohol

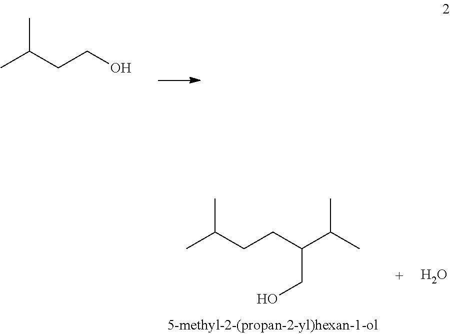

The reaction is carried out under the following conditions:

### Reagents Used

| Isoamyl alcohol 99% | |
|---|---|
| m, g | 75.8 |
| n, mol | 0.86 |
| KOH 85% | |
| m, g | 1.1 |
| % (/alcohol) | 1.5 |
| Catalyst | |
| Name | Pricat Cu 50/8 |
| m, mg | 19.0 |
| % (/alcohol) | 0.025 |

### Operating Conditions

| Fixed parameters | |
|---|---|
| Temperature, ° C. | 180 to 230 |
| Duration, h | 24 |
| Nitrogen bubbling | No |

### Analytical Report of the Synthesis of Guerbet C10 Alcohol

| Crude Product | |
|---|---|
| Composition (CPG) | |
| (see method in | |
| appendix) | |
| Isoamyl alcohol, % | <5% |
| iC10 alcohol, % | >75% |
| Heavy alcohols, % | Max 20% |

Heavier products of the C15 trimer type were obtained as impurities.

Purification by vacuum distillation is therefore necessary.

A topping allows the elimination of isoamyl alcohol traces.

The heavier alcohols are then removed by tailing.

### Analytical Report of the Purification of the Guerbet C10 Alcohol

| Final product | |
|---|---|
| Composition (CPG) | |
| (see method in | |
| appendix) | |
| Isoamyl alcohol, % | <1% |
| iC10 alcohol, % | >94% |
| Heavy alcohols, % | <5% |

### Dehydration of the Guerbet C10 Alcohol

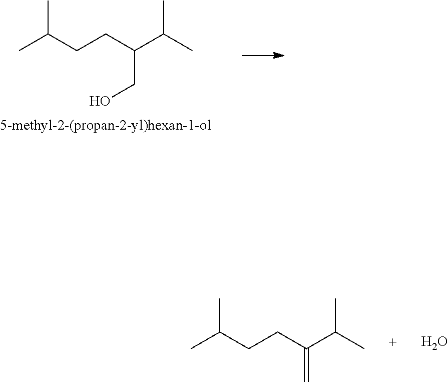

### Composition of the Guerbet C10 Alcohol

| Raw material | Content, % | Supplier |
|---|---|---|
| 5-methyl-2-(propan-2-yl) | Min 94% | BIOSYNTHIS |
| hexan-1-ol | | |

### Raw Materials Used:

| Raw material | Supplier |
|---|---|
| Aluminum | Johnson & Matthey |
| Silica wool | VWR |
| Nitrogen | Air Liquide |

### Dehydration

A catalyst bed is placed at mid-height of the reactor. A thermocouple is placed at the core of this bed. The latter is supported by a grid upon which the silica wool is placed. Above the bed, silica wool is added to block the catalyst bed.

The catalyst used is an industrial alumina supplied by Johnson &

Matthey.

The LHSV (Liquid Hourly Space Velocity) corresponds to the alcohol flow rate passed through the reactor expressed in mL/min/mL of catalyst.

### Dehydration Results

| | | | | | Quantity | Quantity |
|---|---|---|---|---|---|---|
| | | | | | of | of |
| | | | | | water | alkenes |
| | | | | | produced | produced |
| | | | | | per 1 | per 1 |
| | | | d | | ton of | ton of |
| | | | (mL/ | Conver- | alcohol, | alcohol, |
| Alcohol | T,° C. | LHSV | min) | sion | kg | kg |
| Guerbet | 330 | 2.5 | 0.5 | >99% | 114 | 886 |
| C10 | | | | | | |
| alcohol | | | | | | |

### Alkene Characterization

| | | | Appearance at |
|---|---|---|---|
| | Alkene | Alkene iodine value | room |
| Alkene | Formula | g I2/100 g | temperature |
| Alkene ic10, | 94% C10H20 | 161 | Liquid |

### Hydrogenation of the Alkenes

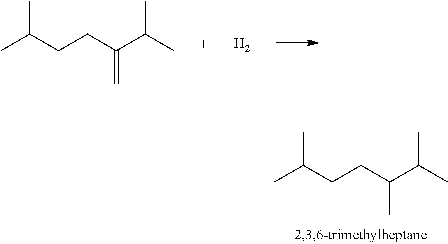

The hydrogenation reaction takes place in a one liter batch reactor.

The operating conditions are detailed below.

The alkene 600 g and the catalyst (Raney nickel) 3 g are introduced into the reactor at room temperature. The reactor is inerted with 3×5 bars of nitrogen and the temperature is raised to the working temperature of 180° C. 5 bars of hydrogen are then introduced. After two hours, the temperature is raised to 200° C. and the pressure to 10 bar. The reaction is then maintained for an additional 3 hours.

### Characterization of the Alkane

| | Hydrogen | | | |
|---|---|---|---|---|
| | consumption | | | Appearance at |
| | per 1 ton of | Alkane | Alkene iodine | room |
| Alkane | alkene, kg H2 | formula | value g I2/100 g | temperature |
| Alkane | 12.7 | C10H22 | <1 | Liquid |
| ic10, | | | | |

### Example C2—Synthesis of 6-methylundecane

Synthesis of Guerbet C12 from Vegetable Hexanolic Alcohol

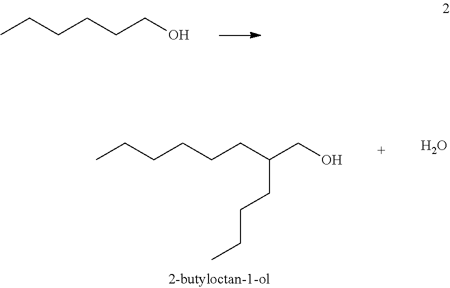

The reaction is carried out under the following conditions

### Reagents Used for the Synthesis of Guerbet C12

| | | |
|---|---|---|
| n-hexanol 99% | m, g | 87.7 |
| | n, mol | 0.86 |
| KOH 85% | m, g | 1.3 |
| | % (/alcohol) | 1.5 |
| Catalyst | | Pricat Cu 50/8 |
| | Name | |
| | m, mg | 22.0 |
| | % (/alcohol) | 0.025 |

### Guerbet C12 Synthesis Operating Conditions

| | |
|---|---|
| Temperature, ° C. | 180 to 230 |
| Duration, h | 24 |
| Nitrogen bubbling | No |

### Analytical Report of the Synthesis of Guerbet C12 Alcohol

| Crude product | |
|---|---|
| Composition (CPG) | |
| (see method in | |
| appendix) | |
| hexanol alcohol, % | <5% |
| 2 butyl octanol, % | >75% |
| Heavy alcohols, % | Max 20% |

Heavier products of the C18 trimer type were obtained as by-products. Purification by vacuum distillation is therefore necessary.

A topping allows the elimination of hexanolic alcohol traces.

The heavier alcohols are then removed by tailing.

### Analytical Report of the Purification of Guerbet C12 Alcohol

| Final product | |
|---|---|
| Composition (CPG) | |
| (see method in appendix) | |
| Isoamyl alcohol, % | <1% |
| 2 butyl octanol, % | >94% |
| Heavy alcohols, % | <5% |

### Dehydration of the Guerbet C12 Alcohol

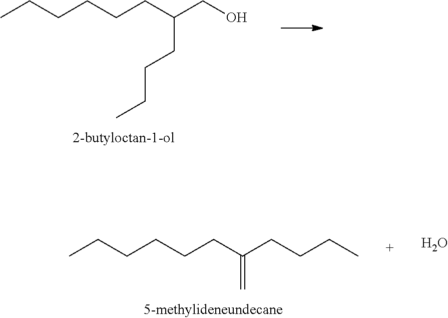

The raw materials used for this study are presented in the following table. No hazardous reagents or solvents are used. The catalyst is an industrial aluminum.

### Composition of the Guerbet Alcohol

| Raw Materials | Content, % | Suppliers |
|---|---|---|
| 2 butyl octanol | Min 94% | BIOSYNTHIS |

### Raw Materials Used:

| Raw Materials. | Suppliers |
|---|---|
| Aluminum | Johnson & |
| | Matthey |
| Silica wool | VWR |
| Nitrogen | Air Liquide |

### Dehydration

Dehydration is carried out according to the procedure described above.

The LHSV (Liquid Hourly Space Velocity) corresponds to the alcohol flow rate passed through the reactor expressed in mL/min/mL of catalyst.

Dehydration Results

| | | | | | Quantity | Quantity |
|---|---|---|---|---|---|---|
| | | | | | of water | of alkenes |
| | | | | | produced | produced |
| | | | | | per | per 1 |
| | | | d | | 1 ton of | ton of |
| | T, | | (mL/ | Conver- | alcohol, | alcohol, |
| Alcohol | ° C. | LHSV | min) | sion | kg | kg |
| Guerbet | 330 | 2.5 | 0.5 | >99% | 96.7 | 903 |
| C12 | | | | | | |
| alcohol | | | | | | |

Alkene Characterization

| | Alkene | Alkene iodine value | Appearance at |
|---|---|---|---|
| Alkene | Formula | g I2/100 g | room temperature |
| Alkene ic12, | 94% C12H24 | 151 | Liquid |

Hydrogenation reaction

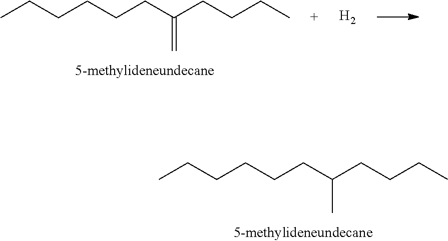

Hydrogenation is carried out according to the procedure described above.

### Characterization of the Alkane

| | Hydrogen | | | |
|---|---|---|---|---|
| | consumption | | | Appearance at |
| | per 1 ton of | Alkane | Alkene iodine | room |
| Alkane | alkene, kg H2 | formula | value g I2/100 g | temperature |
| Alkane ic12 | 12.7 | C12H26 | <1 | Liquid |

1-13. (canceled)

14. A cosmetic excipient comprising a mixture of alkanes, the mixture of alkanes comprising at least one C8-C10 alkane, and at least one C≥11 alkane, wherein the at least one C8-C10 alkane is present in a mass percentage less than or equal to approximately 50% (≤50%), relative to the total mass of the mixture of alkanes, wherein it is entirely obtained from raw materials of plant, bacterial, or animal origin and that the mixture of alkanes is free of alkanes with an odd carbon number.

15. The cosmetic excipient according to claim 14, wherein it is entirely obtained from raw materials of plant origin.

16. The cosmetic excipient comprising a mixture of alkanes according to claim 14, wherein the at least one C8-C10 alkane is selected from the group consisting of linear or branched C8 and C10 alkanes, and mixtures thereof.

17. The cosmetic excipient comprising a mixture of alkanes according to claim 14, wherein the at least one C8-C10 alkane is at least a linear or branched C10 alkane.

18. The cosmetic excipient comprising a mixture of alkanes according to claim 14, wherein the at least one C8-C10 alkane is selected from the group consisting of 4-methylnonane (C10H22, CAS 17301-94-9), 2-methylnonane (C10H22, CAS 871-83-0), n-decane (C10H22, CAS 124-18-5), and mixtures thereof.

19. The cosmetic excipient comprising a mixture of alkanes according to claim 14, wherein the at least one C≥11 alkane is selected from the group consisting of C12, C14, C16, C18, C20, C22, and C24 alkanes, and mixtures thereof.

20. The cosmetic excipient comprising a mixture of alkanes according to claim 14, wherein the at least one C≥11 alkane is selected from the group consisting of branched C12, C14, C16, C18, C20, C22, C24 alkanes, and mixtures thereof.

21. The cosmetic excipient comprising a mixture of alkanes according to claim 14, wherein the at least one C8-C10 alkane is at least a C10 methyl alkane.

22. The cosmetic excipient comprising a mixture of alkanes according to claim 14, wherein the at least one C≥11 alkane is at least a C12 methyl alkane.

23. The cosmetic excipient comprising a mixture of alkanes according to claim 14, wherein the at least one C≥11 alkane is at least a C14 methyl alkane.

24. The cosmetic excipient comprising a mixture of alkanes according to claim 14, wherein the at least one C≥11 alkane is at least a C16 methyl alkane.

25. The cosmetic excipient comprising a mixture of alkanes according to claim 14, wherein the at least one C≥11 alkane is present in a percentage by mass of between approximately 50 and approximately 99% (50%≤percentage by mass≤99%) relative to the total mass of the mixture of alkanes.

26. The cosmetic excipient comprising a mixture of alkanes according to claim 14, the excipient further comprising polysilicone 11 the cosmetic excipient according to claim 14, wherein the alkane mixture has a flash point less than or equal to 69° C. (≤69° C.).

27. The cosmetic excipient according to claim 14, wherein the percentage of evaporation at t=10 hours is between 10 and 30 (10%≤percentage of evaporation at t=10≤30%).
